(19) United States
(12) Patent Application Publication
(10) Pub. No.: US 2012/0237466 A1
(43) Pub. Date: Sep. 20, 2012

(54) PHOTO-FILTERING COMPOSITION FOR HAIR AND SCALP

(76) Inventor: James Graham, Silver Springs, FL (US)

(21) Appl. No.: 13/424,275

(22) Filed: Mar. 19, 2012

Related U.S. Application Data
(60) Provisional application No. 61/454,327, filed on Mar. 18, 2011.

(51) Int. Cl. A61K 8/92; A61Q 5/06; A61K 8/97; A61Q 17/04

(52) U.S. Cl. 424/60; 424/59

(57) ABSTRACT

An all natural topically applied broad spectrum photo-filtering composition for both styling of hair and protecting hair and scalp from ultraviolet radiation is comprised of natural oils, vitamins and amino acids having UV absorption properties which in combination with at least one inorganic particulate selected from the group consisting of titanium dioxide arid zinc oxide afford broad spectrum photo protection of both hair and scalp across the UV and IRA spectrum. Combining the above with bees wax, glycerin, gelatin and wafer, produces an emulsified gel suitable for styling hair while simultaneously protecting the hair and scalp from damaging UV and IR rays.

### RELATED APPLICATION

This application claims the benefit of U.S. Provisional Application No. 61/454,327, filed Mar. 19, 2011 and entitled, Photo Filtering Composition for Hair and Scalp.

### FIELD OF THE INVENTION

The subject invention relates to hair and scalp care generally, and to an all natural topically applied broad spectrum photo filtering composition for both styling of hair and protecting hair and scalp from ultraviolet radiation in particular.

### BACKGROUND OF THE INVENTION

Care of the hair and care of the scalp skin may appear separate, but are actually intertwined because hair grows from beneath the skin. The living parts of hair (hair follicle, hair root, root sheath, and sebaceous gland) are beneath the skin, while the actual hair shaft which emerges (the cuticle which covers the cortex and medulla) has no living processes. Accordingly, scalp skin, just like any other skin on the body, must be kept healthy to ensure a healthy body and healthy hair production. In order to do so, hair and scalp skin must be protected from ultraviolet radiation which is known to damage both.

Ultraviolet (UV) radiation is defined as that portion of the electromagnetic spectrum between x-rays and visible light, i.e., between 40 and 400 nm (30-3 eV). The UV spectrum is divided into Vacuum UV (40-190 nm), Far UV (190-220 nm), UVC (220-290 nm), UVB (290-320), and UVA (320-400 nm). UVC is almost never observed in nature because it is absorbed completely in the atmosphere, as are Far UV and Vacuum UV. In humans, UVC is absorbed in the outer dead layers of the epidermis. Accidental overexposure to UVC can cause corneal burns, commonly termed welders' flash, and snow blindness, a severe sunburn to the face. While UVC injury usually clears up in a day or two, it can be extremely painful. UVB is typically the most destructive form of UV radiation because it has enough energy to cause photochemical damage to cellular DNA, yet not enough to be completely absorbed by the atmosphere. UVB is needed by humans for synthesis of vitamin D; however, harmful effects can include erythema (sunburn), cataracts, and development of skin cancer. Individuals working outdoors are at the greatest risk of UVB effects. Most solar UVB is blocked by ozone in the atmosphere, and there is concern that reductions in atmospheric ozone could increase the prevalence of skin cancer. UVA is the most commonly encountered type of UV light. UVA exposure has an initial pigment-darkening effect (tanning) followed by erythema if the exposure is excessive. Atmospheric ozone absorbs very little of this part of the UV spectrum. UVA is needed by humans for synthesis of vitamin D; however, overexposure to UVA has been associated with toughening of the skin, suppression of the immune system, and cataract formation. For total protection against damage from the sun, the skin needs to be protected from UVA, UVB and IRA (Infra Red Energy). Roughly 35% of solar energy is IRA.

Sunscreens and sun blocks are used to protect the skin from UV radiation. Although they have similar properties and are both important in caring of the skin, sun block is opaque and is stronger than sunscreen since it is able to block a majority of the UVA/UVB rays from the sun, thus not having to be reapplied several times a day. Titanium dioxide and zinc oxide are two of the important ingredients in sun block and are inorganic particulates that reflect, scatter and absorb UV light. Sunscreen is more transparent once applied to the skin and also has the ability to protect against UVA/UVB rays as well, although the sunscreen's ingredients have the ability to break down at a faster rate once exposed to sunlight, and some of the radiation is able to penetrate to the skin. In order for sunscreen to be more effective the user needs to consistently reapply at least every two hours, and use a higher SPF.

Many hair care products include UV filtering agents to shield the hair from damaging rays. Such products include shampoos, conditioners, hair sprays and hair coloring products. Similarly, there exist a myriad of different topically applied lotions, sprays, gels and the like that absorb and/or reflect some of the sun's UV radiation in order to protect the skin. Ironically, none of these skin care products are intended for, or effective at, styling of the hair, and none of the above hair care products are intended for, or effective at, protecting the scalp from UV radiation. Despite all the hair and skin care product available on the market today/there is no single product that is useful as both a hair styling gel or pomade, and as a photo filtering agent to protect both hair and scalp from UV radiation. Clearly, there is a need for such a composition. Moreover, with the onslaught of chemical components recently approved by the FDA for use in sunscreens and sun blocks, many of which have questionable side effects, an all natural alternative should be available.

### SUMMARY OF THE INVENTION

The present invention reduces or completely eliminates the above described shortcomings of the prior art by providing an all natural topically applied broad spectrum photo filtering composition for both styling of hair and protecting hair and scalp from ultraviolet radiation. More specifically, the hair styling and photo filtering compositions of the subject invention are comprised of natural oils, vitamins and amino acids having UV absorption properties which in combination with at least one inorganic particulate selected from the group consisting of titanium dioxide and zinc oxide afford broad spectrum photo protection of both hair and scalp across the UV and IRA spectrum. Combining the above with bees wax, glycerin, gelatin and water, produces an emulsified gel suitable for styling hair while simultaneously protecting the hair and scalp from damaging UV and IR rays.

There has thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is, therefore, a primary object of the subject invention to provide a topical preparation for the hair and scalp wherein the preparation functions to shape and style the hair in the manner of a hair gel or pomade while simultaneously affording both hair and scalp with protection from ultraviolet and infrared radiation.

It is another primary object of the present invention to provide a hair and scalp preparation with the above properties and that is made from natural ingredients.

Another primary object of the present invention is to provide a hair and scalp preparation comprised of photo-filtering ingredients which in combination protect the hair and scalp from a broad spectrum of damaging light frequencies.

Yet another primary object of the present invention is to provide a hair and scalp preparations having the above characteristics and which are easily and inexpensively manufactured.

It is also an object of the present invention to provide photo-filtering compositions having the above described properties and which may be safely used without serious side effects.

These together with other objects of the invention, along with the various features of novelty which characterize the invention, are pointed out with particularity in the claims annexed to and forming a part of this disclosure. For a better understanding of the invention, its advantages and the specific objects attained by its uses, reference should be had to the accompanying drawings and descriptive matter in which there is illustrated a preferred embodiment of the invention.

### DETAILED DESCRIPTION OF THE PREFERRED EMBODIMENT

The present invention is comprised of hair and scalp preparations for mammals generally, and especially for people exposed to damaging electromagnetic radiation of frequencies between approximately 290 nm and 400 nm. The subject preparations are also useful in the styling and shaping of hair.

A first embodiment of the subject hair and scalp preparation is comprised of: a) a lotion component comprised of a solid oil, a liquid oil, an emulsifier and water; b) a hair styling component comprised of gelatin, glycerol and water; and c) a photo-filtering component comprised of vitamins, amino acids and at least one inorganic photo-filtering agent selected from the group consisting of titanium dioxide and zinc oxide. The lotion component serves as a base or carrier for the photo-filtering component and the hair styling component facilitates shaping and holding of the user's hair as well as adding sheen. A variety of additional ingredients may be optionally be added to the above described composition to impart other desirable characteristics and properties as set forth in more detail below.

### EXAMPLE

A first example of the above-described embodiment of the subject hair and scalp preparation is comprised of the following ingredients:

Lotion Component:
1 oz bees wax
1 oz coconut oil
2.5 oz sunflower oil
3.5 oz flax seed oil
2 tbsp petroleum jelly

Hair Styling Component:
0.550 mL water
50 mL glycerol
21 g gelatin

Photo-Filtering Component:
UV 20 g Titanium dioxide
100,000 I.U. Vitamin A
1000 mg Vitamin B2
10000 mcg Vitamin B12
5 ml Vitamin E (32,000 I.U.)
1000 mcg Vitamin K
5000 mg Tryptophan

Non-Essential Ingredients:
20 drops Orange Oil
Sorbic acid

Method of Preparation: The water-soluble components were combined and heated until dissolved. Similarly, the oil-soluble components were combined and heated until dissolved. The dissolved components were then combined together with titanium dioxide and blended until a homogenous creamy substance was formed. The mixture was permitted to cool and blended occasionally to maintain homogeneity. The resulting preparation had the consistency of a pomade which was effective in both styling of hair and filtering both hair and scalp from harmful UV light. Orange essential oil was added as a fragrance. Anyone or combination of known preservatives may also be added to the subject composition, including natural and manmade agents such as Sorbic acid, Sodium sorbate or sorbic acid sodium salt, Potassium sorbate, Calcium sorbate, Benzoic acid, Sodium benzoate, Potassium benzoate, Calcium benzoate, Ethyl 4-hydroxybenzoate, Ethyl 4-hydroxybenzoate sodium salt, Propyl 4-hydroxybenzoate, Sodium salt of Propyl 4-hydroxybenzoate, Methyl 4-hydroxybenzoate, Sodium salt of Methyl 4-hydroxybenzoate, Sulphur dioxide, Sodium sulphite, Sodium hydrogen sulphite, Sodium metabisulphite, Potassium metabisulphite, Potassium sulphite, Calcium sulphite, Calcium hydrogen sulphite, Potassium hydrogen sulphite, Biphenyl, 2-hydroxybiphenyl, Sodium biphenyl-2-yl oxide, 2-(Thiazol-4-yl) benzimidazole, Nisin, Pimaracin, Formic acid, Sodium formate, Calcium formate, Hexamine, Formaldehyde, Dimethylcarbonate, Potassium nitrate, Sodium nitrite, Sodium nitrate, Potassium nitrate, Acetic acid, Potassium acetate, Sodium acetate, Calcium acetate, Lactic acid, Propionic acid, Sodium propionate, Calcium propionate, Potassium propionate, Boric acid, Sodium tetraborate, Carbon dioxide, Malic acid, and/or Fumaric acid.

The two liquid oils, namely sunflower oil and flax seed oil, were selected for their high degree of unsaturation. Coconut oil was selected as the “solid oil”. Bees wax and petroleum jelly were used as the emulsifying agents. A mixture of gelatin, glycerol and water were used to create the hair gel component. The photo-filtering component is comprised of a plurality of vitamins and one amino acid all selected based on their UV absorption index (indices) which in combination substantially cover the UV spectrum between approximately 260 nm (UVC) and 400 nm (UVA).

With reference now being made to Table 1, below, it may be appreciated that each of the photo-filtering, chemical agents has at least one absorption peak or wavelength of maximum electromagnetic absorption. As may be readily apparent, although each peak represents the wavelength of maximum absorption, the chemical also absorbs light of wavelengths both longer and shorter than the peak wavelength as represented by the hash marks to the left and right of each peak value. Accordingly, each chemical agent identified absorbs light over at least one range of wavelengths and each such range will overlap with the range of absorption of at least one other chemical agent identified. These overlapping absorption ranges are additive therefore the degree of absorption in overlapping regions is increased. Ironically, scientists measuring the light absorption properties of chemicals on a spectrometer refer to the light absorption regions on either side of the peak as “noise” as this effect would reduce the efficiency in detecting the individual components in a solution. These overlapping regions of weaker light absorption, however, ensure that the subject composition provides UV protection across a broad absorption spectrum.

Some of the agents used in the lotion component of the subject preparation also absorb light and therefore contribute to its photo-filtering property. Specifically, sunflower oil absorbs light below 240 nm, and flax seed oil has a broad absorption spectrum between 280-290 nm. Furthermore, the addition of the TiO2 acts as a refractor of UV light thereby further preventing penetration of rogue UV wavelengths which may slip through the other agents of the photo-filtering components of the subject preparation.

TABLE 1

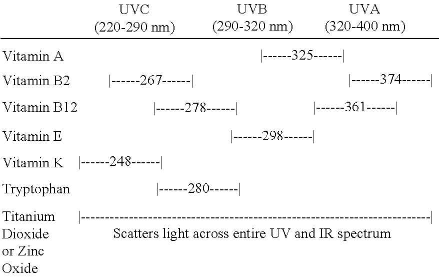

Additional or alternative emulsifying agents may be used to produce the subject hair and scalp preparation including, Calcium Stearoyl Lactylate, Oleth-20, Ceteareth-20, PEG-100 Stearfate, Cetearyl Glucoside, PEG-20 Almond Glycerides, Ceteth-10, PEG-20 Methyl Glucose Sesquistearate, Ceteth-2, PEG-25 Hydrogenated Castor Oil, Ceteth-20, PEG-30 Dipolyhydroxystearate, Cocamide MEA, PEG-4 Dilaurate, Glyceryl Laurate, PEG-40 Sorbitan Peroleate, Glyceryl Stearate, PEG-60 Almond Glycerides, Glyceryl Stearate, PEG-100 Stearate, PEG-8 Laurate, Glyceryl Stearate SE, PEG-80 Sorbitan Laurate, Glycol Distearate, Polysorbate 20, Glycol Stearate, Polysorbate 60, Isoceteth-20, Polysorbate 80, Isosteareth-20, Polysorbate 85, Lauramide DEA, Sodium Stearoyl Lactylate, Laureth-23, Sorbitan Isostearate, Laureth-4, Sorbitan Laurate, Lecithin, Sorbitan Oleate, Lecithin, Sorbitan Sesquioleate, Linoleamide DEA, Sorbitan Stearate, Methyl Glucose Sesquistearate, Sorbitan Stearate, Sucrose Cocoate, Oleth-10, Sorbitan Trioleate, Oleth-10/Polyoxyl 10 Oleyl Ether NF, Stearamide MEA, Oleth-2, Steareth-2, Oleth-20, Steareth-21, Glycol Distearate, Cetearyl Glucoside, Sorbitan Trioleate, Polysorbate 85, Glycol Stearate, Glyceryl Stearate, PEG-100 Stearate, Sorbitan Sesquioleate, Stearamide MEA, Glyceryl Stearate, Oleth-10/Polyoxyl 10 Oleyl Ether NF, Lecithin, Oleth-10, Sorbitan Oleate, Oleth-20, Sorbitan Stearate, Ceteth-10, Sorbitan Isostearate, PEG-8 Laurate, Oleth-2, Cocamide MEA, Steareth-2, Polysorbate 60, Calcium Stearoyl Lactylate, PEG-60 Almond Glycerides, Glyceryl Laurate, Isosteareth-20, Ceteth-2, Lauramide DEA, PEG-30 Dipolyhydroxystearate, Polysorbate 80, Glyceryl Stearate SE, PEG-20 Methyl Glucose Sesquistearate, PEG-4 Dilaurate, Ceteareth-20, Sorbitan Stearate, Sucrose Cocoate, Oleth-20, Methyl Glucose Sesquistearate, Steareth-21, Sodium Stearoyl Lactylate, Ceteth-20, Sorbitan Laurate, Isoceteth-20, PEG-40 Sorbitan Peroleate, Polysorbate 20, Lecithin, Laureth-23, Laureth-4, PEG-100 Stearate, PEG-20 Almond Glycerides, PEG-80 Sorbitan Laurate, Linoleamide DEA, and/or PEG-25 Hydrogenated Castor Oil.

Similarly, additional or alternative oils and waxes may be used to produce the subject hair and scalp preparation including, Aleurites Moluccana Seed Oil, Grape (Vitis Vinifera) Seed Oil, Almond Oil NF, Hybrid Safflower(Carthamus Tinctorius) Oil, Anhydrous Lanolin USP, Isopropyl Myristate, Apricot Kernel Oil, Isopropyl Palmitate, Avocado (Persea Gratissima) Oil, Jojoba (Buxus Chinensis) Oil, Babassu Oil, Lanolin, Beeswax, Macadamia (Ternifolia) Nut Oil, Borage (Borago Officinalis) Seed Oil, Mangifera Indica (Mango) Seed Butter, Brazil Nut Oil, Mineral Oil, C12-15 Alkyl Benzoate, Myristyl Myristate, Cannabis Sativa Seed Oil, Olive (Olea Europaea) Oil, Canola Oil, Oryza Sativa (Rice Bran) Oil, Caprylic/Capric Triglyceride, Peanut Oil NF, Carrot (Daucus Carota Sativa) Seed Oil, Petrolatum, Castor (Ricinus Communis) Oil, PPG-15 Stearyl Ether, Ceresin, Retinyl Palmitate, Cetearyl Alcohol, Safflower (Carthamus Tinctorius) Oil, Cetyl Alcohol, Sesame (Sesamum Indicum) Oil, Cetyl Esters, Shea Butter (Butyrospermum Parkii), Cetyl Palmitate, Soybean (Glycine Soja) Oil, Coconut Oil, Stearic Acid, Daucus Carota Sativa (Carrot) Root Extract, Stearyl Alcohol, Diisopropyl Adipate, Sunflower (Helianthus Annus) Oil, Dimethicone, Sweet Almond (Prunus Amygdalus Dulcis) Oil, Dog Rose (Rosa Canina) Hips Oil, Theobroma Cacao (Cocoa) Seed Butter, Emu Oil, Tocopherol, Evening Primrose Oil, and/or Flax Seed Oil.

Furthermore, additional or alternative photo-filtering agents may be incorporated into the subject hair and scalp preparation including any chemically enhanced broad spectrum UV absorber currently used in commercially available sun blocks and sunscreens such as p-Aminobenzoic acid, PABA, Padimate, OD-PABA, octyldimethyl-PABA, σ-PABA, Phehylbenzimidazole sulfonic acid, Ensulizole, Eusolex 232, PBSA, Parsol HS, Cinoxate, 2-Ethoxyethyl p-methoxycinnamate, Dioxybenzone, Benzophenone-8, Oxybenzone, Benzophenone-3, Eusolex 4360, Escalol 567, Homosalate, Homomethyl salicylate, HMS, Menthyl anthranilate, Meradimate, Octocrylene, Eusolex OCR, 2-cyano-3,3diphenyl acrylic acid, 2-ethylhexylester, Octyl methoxycinnamate, Octinoxate, EMC, OMC, Ethylmethoxycinnamate, Escalol 557, 2-ethylhexyl-paramethoxycinnamate, Parsol MCX, Octyl salicylate, Octisalate, 2-Ethylhexyl salicylate, Escalol 587, Sulisobenzone, 2-Hydroxy-4-Methoxybenzophenone-5-sulfonic acid, 3-benzoyl-4-hydroxy-6-methoxybenzenesulfonic acid, Benzophenone-4, Escalol 577, Trylamine salicylate, Triethanolamine salicylate, Avobenzone, 1-(4-methoxyphenyl)-3-(4-tert-butylphenyl)propane-1,3-dione, Butyl methoxy dibenzoylmethane, BMDBM, Parsol 1789, Eusolex 9020, Ecamsule, Mexoryl SX, Terephthalylidene Dicamphor Sulfonic Acid, Titanium dioxide, CI77891, Zinc oxide, 4-Methylbenzylidene camphor, Enzacamene, Parsol 5000, Eusolex 6300, MBC, Tinosorb M, Bisoctrizole, Methylene Bis-Benzotriazolyl Tetramethylbutylphenol, MBBT, Tinosorb S, Bis-ethylhexyloxyphenol methoxyphenol triazine, Bemotrizinol, BEMT, anisotriazine, Neo Heliopan AP, Bisdisulizole Disodium, Disodium phenyl dibenzimidazole tetrasulfonate, bisimidazylate, DPDT, Mexoryl XL, Drometrizole Trisiloxane, Benzophenone-9, Uvinul DS 49, CAS 3121-60-6, Sodium Dihydroxy Dimethoxy Disulfobenzophenone, Uvinul T 150, Octyl triazone, ethylhexyl triazone, EHT, Uvinul A Plus, Diethylamino Hydroxybenzoyl Hexyl Benzoate, Uvasorb HEB, Iscotrizinol, Diethylhexyl butamido triazone, DBT, Parsol SLX, Dimethico-diethylbenzalmalonate, Polysilicone-15, Isopentenyl-4-methoxycinnamate, Isoamyl p-Methoxycinnamate, IMC, Neo Heliopan E1000, Amiloxate, Isoamyl methoxycinnamate, 4-Methylbenzylidene camphor, and/or Octyl triazone.

An alternate embodiment of the subject hair and scalp preparation may include insect repelling agents including, DEET (N,N-diethyl-m-toluamide), Essential oil of the lemon eucalyptus (Corymbia citriodora), p-methane-3,8-diol (PMD), Icaridin, picaridin, bayrepel, KBR 3023, Nepetalactone, catnip oil, Citronella oil, Permethrin, Neem oil, Bog Myrtle, Achillea alpina, alpha-terpinene, Basil, Callicarpa Americana, Camphor, Carvacrol, Castor oil, Cedar oil, Celery extract, Clove oil, Cinnamon, Eucalyptus oil, Fennel oil, Garlic, Geranium oil, Lavender, Lemongrass oil, Marigolds, Marjoram, Oleic acid, Peppermine, Pennroyal, Pyrethrum, Rosemary, Lantana camara, Solanum villosum, tea tree, and/or thyme.

Yet another embodiment of the subject hair and scalp preparation may include pheromones including, all allomones, Androstadienone, and/or Estratetraenol.

The above described compositions may be used to protect not only humans, but other mammals including domestic animals such as dogs and horses and the like. For instance, white colored horses tend to get sunburn around their eyes, eyelids, lips and nose where hair growth is minimal and there is a lack of pigment. Moreover, because embodiments of the subject compositions consist of all-natural ingredients, they are well suited for infants and children as well.

Although the present invention has been described with reference to the particular embodiments herein set forth, it is understood that the present disclosure has been made only by way of example and that numerous changes in details of construction may be resorted to without departing from the spirit and scope of the invention. Thus, the scope of the invention should not be limited by the foregoing specifications, but rather only by the scope of the claims appended hereto.

What is claimed as being new, useful and desired to be protected by Letters Patent of the United States is as follows:

1. A broad spectrum photo-filtering composition for both styling of hair and protecting hair and scalp from ultraviolet radiation, comprising: a) a lotion component comprised of at least one oil, an emuisifier and water; b) a hair styling component comprised of gelatin, glycerol, and water; and c) a photo-filtering component comprised of 1) a plurality of vitamins and at least one amino acid together having UV indices substantially covering the UV spectrum between approximately 260 nm (UVC) and 400 nm (UVA), and 2) at least one inorganic photo-filtering agent selected from the group consisting of titanium dioxide and zinc oxide, whereby the lotion component serves as a base or carrier for the photo-filtering component and the hair styling component facilitates shaping and holding of the user's hair as well as adding sheen thereto.

2. The broad spectrum photo-filtering composition of claim 1, wherein said plurality of vitamins is comprised of Vitamin A, Vitamin B2, Vitamin B12, Vitamin E and Vitamin K.

3. The broad spectrum photo-filtering composition of claim 1, wherein said at least one amino acid is tryptophan.

4. The broad spectrum photo-filtering composition of claim 2, wherein said at least one amino acid is tryptophan.

5. The broad spectrum photo-filtering composition of claim 4, wherein said at least one oil is comprised of the solid oil, coconut oil, and the liquid oils, flax seed oil and sunflower oil, and said emuisifier is comprised of bees wax and petroleum jelly.

6. The broad spectrum photo-filtering composition of claim 4, wherein said at least one oil is selected from the group consisting of Aleurites Moluccana Seed Oil, Grape (Vitis Vinifera) Seed Oil, Almond Oil NF, Hybrid Safflower (Carthamus Tinctorius) Oil, Anhydrous Lanolin USP, Isopropyl Myristate, Apricot Kernel Oil, Isopropyl Palmitate, Avocado (Persea Gratissima) Oil, Jojoba (Buxus Chinensis) Oil, Babassu Oil, Lanolin, Beeswax, Macadamia (Ternifolia) Nut Oil, Borage (Borago Officinalis) Seed Oil, Mangifera Indica (Mango) Seed Butter, Brazil Nut Oil, Mineral Oil, C12-15 Alkyl Benzoate, Myristyl Myristate, Cannabis Sativa Seed Oil, Olive (Olea Europaea) Oil, Canola Oil, Oryza Sativa (Rice Bran) Oil, Caprylic/Capric Triglyceride, Peanut Oil NF, Carrot (Daucus Carota Sativa) Seed Oil, Petrolatum, Castor (Ricinus Communis) Oil, PPG-15 Stearyl Ether, Ceresin, Retinyl Palmitate, Cetearyl Alcohol, Safflower (Carthamus Tinctorius) Oil, Cetyl Alcohol, Sesame (Sesamum Indicum) Oil, Cetyl Esters, Shea Butter (Butyrospermum Parkii), Cetyl Palmitate, Soybean (Glycine Soja) Oil, Coconut Oil, Stearic Acid, Daucus Carota Sativa (Carrot) Root Extract; Stearyl Alcohol, Diisopropyl Adipate, Sunflower (Helianthus Annus) Oil, Dimethicone, Sweet Almond (Prunus Amygdalus Dulcis) Oil, Dog Rose (Rosa Canina) Hips Oil, Theobroma Cacao (Cocoa) Seed Butter, Emu Oil, Tocopherol, Evening Primrose Oil, and Flax Seed Oil.

7. The broad spectrum photo-filtering composition of claim 4, wherein said emulsifier is comprised of at least one emulsifying agent selected from the group consisting of Calcium Stearoyl Lactylate, Oleth-20, Ceteareth-20, PEG-100 Stearate, Cetearyl Glucoside, PEG-20 Almond Glycerides, Ceteth-10, PEG-20 Methyl Glucose Sesquistearate, Ceteth-2, PEG-25 Hydrogenated Castor Oil, Ceteth-20, PEG-30 Dipolyhydroxystearate, Cocamide MEA, PEG-4 Dilaurate, Glyceryl Laurate, PEG-40 Sorbitan Peroleate, Glyceryl Stearate, PEG-60 Almond Glycerides, Glyceryl Stearate, PEG-100 Stearate, PEG-8 Laurate, Glyceryl Stearate SE, PEG-80-Sorbitan Laurate, Glycol Distearate, Polysorbate 20, Glycol Stearate, Polysorbate 60, Isoceteth-20, Polysorbate 80, Isosteareth-20, Polysorbate 85, Lauramide DEA, Sodium Stearoyl Lactylate, Laureth-23, Sorbitan Isostearate, Laureth-4, Sorbitan Laurate, Lecithin, Sorbitan Oleate, Lecithin, Sorbitan Sesquioleate, Linoleamide DEA, Sorbitan Stearate, Methyl Glucose Sesquistearate, Sorbitan Stearate, Sucrose Cocoate, Oleth-10, Sorbitan Trioleate, Oleth-10/Polyoxyl 10 Oleyl Ether NF, Stearamide MEA, Oleth-2, Steareth-2, Oleth-20, Steareth-21, Glycol Distearate, Cetearyl Glucoside, Sorbitan Trioleate, Polysorbate 85, Glycol Stearate, Glyceryl Stearate, PEG-100 Stearate, Sorbitan Sesquioleate, Stearamide MEA, Glyceryl Stearate, Oleth-10/Polyoxyl 10 Oleyl Ether NF, Lecithin, Oleth-10, Sorbitan Oleate, Oleth-20, Sorbitan Stearate, Ceteth-10, Sorbitan Isostearate, PEG-8 Laurate, Oleth-2, Cocamide MEA, Steareth-2, Polysorbate 60, Calcium Stearoyl Lactylate, PEG-60 Almond Glycerides, Glyceryl Laurate, Isosteareth-20, Ceteth-2, Lauramide DEA, PEG-30 Dipolyhydroxystearate, Polysorbate 80, Glyceryl Stearate SE, PEG-20 Methyl Glucose Sesquistearate, PEG-4 Dilaurate, Ceteareth-20, Sorbitan Stearate, Sucrose Cocoate, Oleth-20, Methyl Glucose Sesquistearate, Steareth-21, Sodium Stearoyl Lactylate, Ceteth-20, Sorbitan Laurate, Isoceteth-20, PEG-40 Sorbitan Peroleate, Polysorbate 20, Lecithin, Laureth-23, Laureth-4, PEG-100 Stearate, PEG-20 Almond Glycerides, PEG-80 Sorbitan Laurate, Linoleamide DEA, and PEG-25 Hydrogenated Castor Oil.

8. The broad spectrum photo-filtering composition of claim 6, wherein said emuisifier is comprised of at least one, emulsifying agent selected from the group consisting of Calcium Stearoyl Lactylate, Oleth-20, Ceteareth-20, PEG-100 Stearate, Cetearyl Glucoside, PEG-20 Almond Glycerides, Ceteth-10, PEG-20 Methyl Glucose Sesquistearate, Ceteth-2, PEG-25 Hydrogenated Castor Oil, Ceteth-20, PEG-30 Dipolyhydroxystearate, Cocamide MEA, PEG-4 Dilaurate, Glyceryl Laurate, PEG-40 Sorbitan Peroleate, Glyceryl Stearate, PEG-60 Almond Glycerides, Glyceryl Stearate, PEG-100 Stearate, PEG-8 Laurate, Glyceryl Stearate SE, PEG-80 Sorbitan Laurate, Glycol Distearate, Polysorbate 20, Glycol Stearate, Polysorbate 60, Isoceteth-20, Polysorbate 80, Isosteareth-20, Polysorbate 85, Lauramide DEA, Sodium Stearoyl Lactylate, Laureth-23, Sorbitan Isostearate, Laureth-4, Sorbitan Laurate, Lecithin, Sorbitan Oleate, Lecithin, Sorbitan Sesquioleate, Linoleamide DEA, Sorbitan Stearate, Methyl Glucose Sesquistearate, Sorbitan Stearate, Sucrose Cocoate, Oleth-10, Sorbitan trioleate, Oleth-10/Polyoxyl 10 Oleyl Ether NF, Stearamide MEA, Oleth-2, Steareth-2, Oleth-20, Steareth-21, Glycol Distearate, Cetearyl Glucoside, Sorbitan Trioleate, Polysorbate 85, Glycol Stearate, Glyceryl Stearate, PEG-100 Stearate, Sorbitan Sesquioleate, Stearamide MEA, Glyceryl Stearate, Oleth-10/Polyoxyl 10 Oleyl Ether NF, Lecithin, Oleth-10, Sorbitan Oleate, Oleth-20, Sorbitan Stearate, Ceteth-10, Sorbitan Isostearate, PEG-8 Laurate, Oleth-2, Cocamide MEA, Steareth-2, Polysorbate 60, Calcium Stearoyl Lactylate, PEG-60 Almond Glycerides, Glyceryl Laurate, Isosteareth-20, Ceteth-2, Lauramide DEA, PEG-30 Dipolyhydroxystearate, Polysorbate 80, Glyceryl Stearate SE, PEG-20 Methyl Glucose Sesquistearate, PEG-4 Dilaurate, Ceteareth-20, Sorbitan Stearate, Sucrose Cocoate, Oleth-20, Methyl Glucose Sesquistearate, Steareth-21, Sodium Stearoyl Lactylate, Ceteth-20, Sorbitan Laurate, Isoceteth-20, PEG-40 Sorbitan Perorate, Polysorbate 20, Lecithin, Laureth-23, Laureth-4, PEG-100 Stearate, PEG-20 Almond Glycerides, PEG-80 Sorbitan Laurate, Linoleamide DEA, and PEG-25 Hydrogenated Castor Oil.

9. The broad spectrum photo-filtering composition of claim 8, further including at least one chemically enhanced broad spectrum UV absorber selected from the group consisting of p-Aminobenzoic acid, PABA, Padimate, OD-PABA, octyldimethyl-PABA, σ-PABA, Phenylbenzimidazole sulfonic acid, Ensulizole, Eusolex 232, PBSA, Parsol HS, Cinoxate, 2-Ethoxyethyl p-methoxycinnamate, Dioxybenzone, Benzophenone-8, Oxybenzone, Benzophenone-3, Eusolex 4360, Escalol 567, Homosalate, Homomethyl salicylate, HMS, Menthyl anthranilate, Meradimate, Octocrylene, Eusolex OCR, 2-cyano-3,3diphenyl acrylic acid, 2-ethylhexylester, Octyl methoxycinnamate, Octinoxate, EMC, OMC, Ethylmethoxycinnamate, Escalol 557, 2-ethylhexyl-paramethoxycinnamate, Parsol MCX, Octyl salicylate, Octisalate, 2-Ethylhexyl salicylate, Escalol 587, Sulisobenzone, 2-Hydroxy-4-Methoxybenzophenone-5-sulfonic acid, 3-benzoyl-4-hydroxy-6-methoxybenzenesulfonic acid, Benzophenone-4, Escalol 577, Trylamine salicylate, Triethanolamine salicylate, Avobenzone, 1-(4-methoxyphenyl)-3-(4-tert-butylphenyl)propane-1,3-dione, Butyl methoxy dibenzoylmethane, BMDBM, Parsol 1789, Eusolex 9020, Ecamsule, Mexoryl SX, Terephthalylidene Dicamphor Sulfonic Acid, Titanium dioxide, CI77891, Zinc oxide, 4-Methylbenzylidene camphor, Enzacamene, Parsol 5000, Eusolex 6300, MBC, Tinosorb M, Bisoctrizole, Methylene Bis-Benzotriazolyl Tetramethylbutylphenol, MBBT, Tinosorb S, Bis-ethylhexyloxyphenol methoxyphenol triazine, Bemotrizinol, BEMT, anisotriazine, Neo Heliopan AP, Bisdisulizole Disodium, Disodium phenyl dibenzimidazole tetrasulfonate, bisimidazylate, DPDT, Mexoryl XL, Drometrizole Trisiloxane, Benzophenone-9, Uvinul DS 49, CAS 3121-60-6, Sodium Dihydroxy Dimethoxy Disulfobenzophenone, Uvinul T 150, Octyl triazone, ethylhexyl triazone, EHT, Uvinul A Plus, Diethylamino Hydroxybenzoyl Hexyl Benzoate, Uvasorb HEB, Iscotrizinol, Diethylhexyl butamido triazone, DBT, Parsol SLX, Dimethico-diethylbenzalmalonate, Polysilicone-15, Isopentenyl-4-methoxycinnamate, Isoamyl p-Methoxycinnamate, IMC, Neo Heliopan E1000, Amiloxate, Isoamyl methoxycinnamate, 4-Methylbenzylidene camphor, and Octyl triazone.

10. The broad spectrum photo-filtering composition of claim 9, further including at least one insecticide and/or repelling agent selected from the group consisting of, DEET (N,N-diethyl-m-toluamide), Essential oil of the lemon eucalyptus (Corymbia citriodora), p-methane-3,8-diol (PMD), Icaridin, picaridin, bayrepel, KBR 3023, Nepetalactone, catnip oil, Citronella oil, Permethrin, Neem oil, Bog Myrtle, Achillea alpina, alpha-terpinene, Basil, Callicarpa Americana, Camphor, Carvacrol, Castor oil, Cedar oil, Celery extract, Clove oil, Cinnamon, Eucalyptus oil, Fennel oil, Garlic, Geranium oil, Lavender, Lemongrass oil, Marigolds, Marjoram, Oleic acid, Peppermine, Pennyroyal, Pyrethrum, Rosemary, Lantana camara, Solanum villosum, tea tree oil and thyme.

11. The broad spectrum photo-filtering composition of claim 9, further including at least one preservative, selected from the group consisting of Sorbic acid, Sodium sorbate or sorbic acid sodium salt, Potassium sorbate, Calcium sorbate, Benzoic acid, Sodium benzoate, Potassium benzoate, Calcium benzoate, Ethyl 4-hydroxybenzoate, Ethyl 4-hydroxybenzoate sodium salt, Propyl 4-hydroxybenzoate, Sodium salt of Propyl 4-hydroxybenzoate, Methyl 4-hydroxybenzoate, Sodium salt of Methyl 4-hydroxybenzoate, Sulphur dioxide, Sodium sulphite, Sodium hydrogen sulphite, Sodium metabisulphite, Potassium metabisulphite, Potassium sulphite, Calcium sulphite, Calcium hydrogen sulphite, Potassium hydrogen sulphite, Biphenyl, 2-hydroxybiphenyl, Sodium biphenyl-2-yl oxide, 2-(Thiazol-4-yl) benzimidazole, Nisin, Pimaracin, Formic acid, Sodium formate, Calcium formate, Hexamine, Formaldehyde, Dimethylcarbonate, Potassium nitrate, Sodium nitrite, Sodium nitrate, Potassium nitrate, Acetic acid, Potassium acetate, Sodium acetate, Calcium acetate, Lactic acid, Propionic acid, Sodium propionate, Calcium propionate, Potassium propionate, Boric acid, Sodium tetraborate, Carbon dioxide, Malic acid, and Fumaric acid.

12. The broad spectrum photo-filtering composition of claim 10, further including at least one preservative selected from the group consisting of Sorbic acid, Sodium sorbate or sorbic acid sodium salt, Potassium sorbate, Calcium sorbate, Benzoic acid, Sodium benzoate, Potassium benzoate, Calcium benzoate, Ethyl 4-hydroxybenzoate, Ethyl 4-hydroxybenzoate sodium salt, Propyl 4-hydroxybenzoate, Sodium salt of Propyl 4-hydroxybenzoate, Methyl 4-hydroxybenzoate, Sodium salt of Methyl 4-hydroxybenzoate, Sulphur dioxide, Sodium sulphite, Sodium hydrogen sulphite, Sodium metabisulphite, Potassium metabisulphite, Potassium sulphite, Calcium sulphite, Calcium hydrogen sulphite, Potassium hydrogen sulphite, Biphenyl, 2-hydroxybiphenyl, Sodium biphenyl-2-yl oxide, 2-(Thiazol-4-yl) benzimidazole, Nisin, Pimaracin, Formic acid, Sodium formate, Calcium formate, Hexamine, Formaldehyde, Dimethylcarbonate, Potassium nitrate, Sodium nitrite, Sodium nitrate, Potassium nitrate, Acetic acid, Potassium acetate, Sodium acetate, Calcium acetate, Lactic acid, Propionic acid, Sodium propionate, Calcium propionate, Potassium propionate, Boric acid, Sodium tetraborate, Carbon dioxide, Malic acid, and Fumaric acid.

13. The broad spectrum photo-filtering composition of claim 9, further including at least one pheromone selected from the group consisting of an allomone, Androstadienone, and Estratetraenol.

14. The broad spectrum photo-filtering composition of claim 10, further including at least one pheromone selected from the group consisting of an allomone, Androstadienone, and Estratetraenol.

15. The broad spectrum photo-filtering composition of claim 11, further including at least one pheromone selected from the group consisting of an allomone, Androstadienone, and Estratetraenol.

16. The broad spectrum photo-filtering composition of claim 12, further including at least one pheromone selected from the group consisting of an allomone, Androstadienone, and Estratetraenol.

17. A broad spectrum photo-filtering composition for both styling of hair and protecting hair and scalp from ultraviolet radiation, comprising: a) a lotion component comprised of coconut oil, sunflower oil, flax seed oil, bees wax, petroleum jelly and water; b) a hair styling component comprised of gelatin, glycerol and water; and c) a photo-filtering component comprised of 1) Vitamin A, Vitamin B2, Vitamin B12, Vitamin E, Vitamin K and tryptophan together having UV indices substantially covering the UV spectrum between approximately 260 nm (UVC), and 400 nm (UVA), and 2) at least one inorganic photo-filtering agent, selected from the group consisting of titanium dioxide and zinc oxide, whereby the lotion component serves as a base or carrier for the photo-filtering component and the hair styling component facilitates shaping and holding of the user's hair as well as adding sheen thereto.
